(19) United States
(12) Patent Application Publication
(10) Pub. No.: US 2003/0103866 A1
(43) Pub. Date: Jun. 5, 2003

(54) Safe mailbox

(76) Inventors: Jian Chun Wang, North Potomac, MD (US); Xu Cong Wang, North Potomac, MD (US)

Correspondence Address: JIAN CHUN WANG, 14001 Saddle River Dr., North Potomac, MD, 20878, US

(21) Appl. No.: 10/001,049

(22) Filed: Dec. 4, 2001

(51) Int. Cl. A61L 2/10

(52) U.S. Cl. 422/24; 422/1

(57) ABSTRACT

Biological weapons are any infectious agent such as a bacteria or virus when used intentionally to inflict harm upon others. This definition is often expanded to include biologically-derived toxins and poisons. Biological warfare agents include both living microorganisms (bacteria, protozoa, rickettsia, viruses, and fungi), and toxins (chemicals) produced by microorganisms, plants, or animals. Ultraviolet (UV) light, which at select wavelengths of energy, is immensely germicidal. Chemicals also denature proteins and nucleic acids of all bacterial and virus. Set UV light and chemicals as disinfection's tool which against bacterial and viruses in inside of Mailbox to secure mails and posters which delivered by outside will be disinfected. The Safe Mailbox is a very important tool that protect innocent people from bioterrorism at individual family level.

### BACKGROUND OF INVENTION

The threat of bioterrorism has heightened over the past few years. Biological weapons are any infectious agent such as a bacteria or virus when used intentionally to inflict harm upon others. This definition is often expanded to include biologically-derived toxins and poisons. Biological warfare agents include both living microorganisms (bacteria, protozoa, rickettsia, viruses, and fungi), and toxins (chemicals) produced by microorganisms, plants, or animals. It is became very important issues that protect innocent people from bioterrorism. At individual family level, the mailbox is a most danger place to keep the mail or poster which been contaminated by the bacterial or virus from outside of family by post office or commercial companies. Ultraviolet (UV) light, which at select wavelengths of energy, is immensely germicidal. It can kill all bacterial and viral particles. Disinfection refers to the reduction of pathogens (disease causing organisms) while sanitation refers to the quality of cleanliness. Reducing the load of pathogens in the environment of your flock will decrease the risk of disease. Disinfectants are chemical agents that can kill pathogens on contact. The use of UV disinfection tool and chemical disinfection tools to make a safer mailbox carrier become more important way to protect individual family from bioterrorism attack in the future.

### BRIEF DESCRIPTION OF THE DRAWINGS

FIG. 1 is a detail side view of the Safe Mailbox.

FIG. 2 is a detail front view of the Safe Mailbox. The Mail Cell Holder can make sure the mail object has a distance with others.

### DETAILED DESCRIPTION

Ultraviolet light is the portion of the spectrum with wavelengths of 100-400 nm. The UV spectrum is further divided into three different types according to wavelength. UVA extends from 320 to 400 nm. UVB extends from 290 to 320 nm, whereas UVC from 100 to 290 nm. UV light damages living cells by directly and indirectly altering nucleic acids, particularly DNA. It cross-link DNA forming pyrimidine. The damage effects of UV on nucleic acids include photodimerizations between adjacent pyrimidine bases, photohydration of cytosine, and, at much lower frequencies, inter- and intra-strand cross-links, protein-DNA cross-links, and rare base adducts. Indirect effects result when reactive oxygen species (ROS) are generated. They also react with DNA, damaging bases, breaking strands, and cross-linking DNA and proteins.

The wavelength dependence for the induction of direct DNA damage by UV light corresponds to the DNA absorbance spectrum. DNA absorbance is maximal and fairly constant across the UVC (100-290 nm) range, then falls through more than three orders of magnitude in the UVB (290-320 nm) and reaches near-negligible levels in the UVA. UVB is responsible for both direct and indirect DNA damage, while UVA produces only indirect damage in microorganisms exposed to sunlight. UVC has most strong effect for directly damaging DNA. Chemicals also denature proteins and nucleic acids of all bacterial and virus. Some are gaseous (e.g. ethylene oxide) and some are effective as aqueous solutions (e.g. bleach). Some may be used in both forms (e.g. formaldehyde). They inactivate vegetative bacteria, fungi, lipid and non-lipid viruses, HBV, TB, Coxiella burnetii, and bacterial spores.

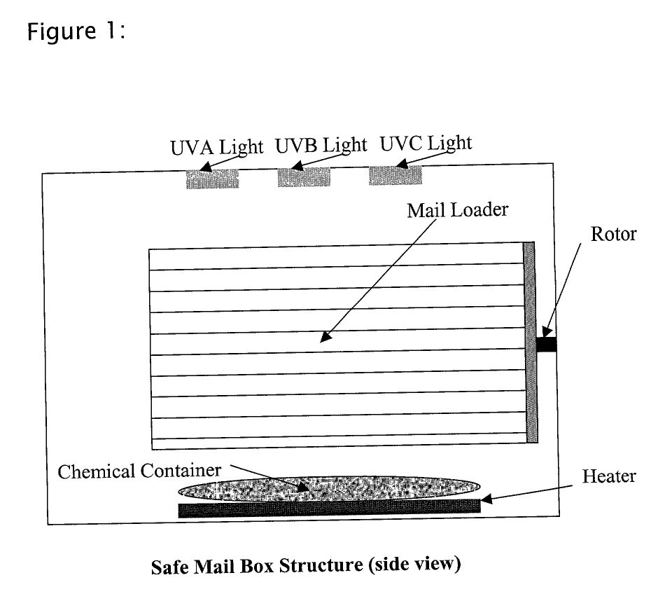

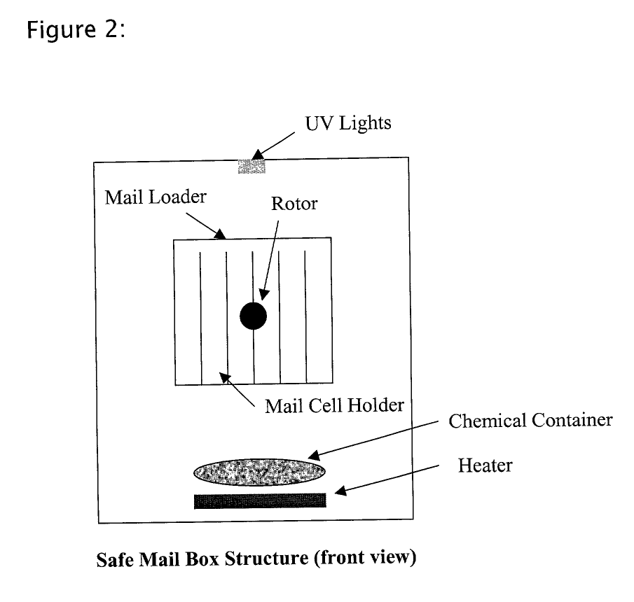

Design a mailbox that has multiple UV light lamps with different wavelength, chemical container with heater at inside. The owner can use safe mailbox to disinfect surface and inside of mails and posters by UV and chemical gas when mails or posters are delivered to mailbox by post office or individual. The Safe Mail Box structure is in FIG. 1 and FIG. 2.

### PARAMETERS

Mailbox size: 21×7×9 inch

UV lamp: 15 Watt (134 nm ultraviolet with 0-15 minute automatic timer)

15 Watt (290 nm ultraviolet with 0-15 minute automatic timer)

15 Watt (370 nm ultraviolet with 0-15 minute automatic timer)

Rotor: 360-degree visibility.

Mailbox wall: 10 mm acrylic material which ensures no UV leakage.

Chemical container: keep disinfect chemical solution.

Disinfect chemical: peracetic acid 3%;

formaldehyde 10%;

sodium hypochlorite 0.5%;

ethylene oxide 30%.

Heater: generate heat up to 70° C. for chemical container.

Door automation: Prevention of harmful UV exposure and chemical gas to personnel is achieved by an automatic shut off mechanism if the front of the mailbox is opened.

Under this condition, all bacterial and viral particles which attached to inside and outside of regular mail (less than three papers in mail) will be killed.

What is claimed is:

1. A method of disinfecting or cleaning and disinfecting objects at mailbox with UV disinfect method and chemical disinfect method.

2. The safe mailbox of claim 1, wherein use ultraviolet light disinfect characters which it cross-link DNA forming pyrmidine to kill all of bacterial, fungi and virus which include vegetative bacteria, fungi, lipid and non-lipid viruses, HBV, TB, Coxiella burnetii, and anthrax spores at surface and inside of object what it is delivered from outside.

3. The safe mailbox of claim 1, wherein use chemical material to disinfect the object at inside of mailbox which infected by bacterial, fungi and virus include vegetative bacteria, fungi, lipid and non-lipid viruses, HBV, TB, Coxiella burnetii, and anthrax spores.

4. The safe mailbox of claim 1, wherein use varied UV wavelengths (UVA, UVB and UVC) to kill bacterial, fungi and virus by direct DNA damage and indirect DNA damage functions on surface and inside of object which is delivered from outside.

5. The safe mailbox of claim 1, wherein use disinfect chemical gaseous characters which generated by temperature increasing to penetrat into inside of object, kill bacterial, fungi and virus at inside of object which is delivered from outside.

6. The safe mailbox of claim 1, wherein use disinfect chemical gaseous characters which generated by temperature rising to kill bacterial, fungi and virus at the surface of object which is delivered from outside.

7. The safe mailbox of claim 1, wherein disinfect chemicals include: chlorine (like: sodium hypochlorite et al), formaldehyde, glutaraldehyde, hydrogen peroxide, alcohols, povidone iodine, ethylene oxide.

8. The safe mailbox of claim 1, wherein the objects which is delivered from outside can be expose by UV light by 360-degree visibility at inside of mailbox.

9. The safe mailbox of claim 1, wherein prevent the leaking of UV light and chimecal gas from mailbox during the disinfect processing.
